(19) United States
(12) Patent Application Publication
(10) Pub. No.: US 2023/0073947 A1
(43) Pub. Date: Mar. 9, 2023

(54) A METHOD FOR PREPARING BOVINE MYOGLOBIN USING ESCHERICHIA COLI

(71) Applicant: iNtRON Biotechnology, Inc., Seongnam-si, Gyeonggi-do (KR)

(72) Inventors: Seong Jun YOON, Seoul (KR); Sang Hyeon KANG, Seoul (KR); Soo Youn JUN, Seoul (KR); An Sung KWON, Gwangju-si Gyeonggi-do (KR); Eun Ji LEE, Seongnam-si Gyeonggi-do (KR)

(21) Appl. No.: 17/791,568

(22) PCT Filed: Jan. 9, 2021
(86) PCT No.: PCT/IB2021/050138

§ 371 Date: Jul. 8, 2022

Related U.S. Application Data
(60) Provisional application No. 62/959,708, filed on Jan. 10, 2020.

(51) Int. Cl. C07K 14/805; A23L 27/26; C12N 15/52; A23L 33/16; A23L 33/18; A61P 7/06

(52) U.S. Cl. CPC C07K 14/805; A23L 27/26; C12N 15/52; C12Y 499/01001; C12Y 101/0104; A23L 33/16; A23L 33/18; A61P 7/06

(57) ABSTRACT

A method for preparing a bovine myoglobin includes: constructing a first plasmid containing genes for heme biosynthesis pathway enzymes; constructing a second plasmid containing a gene for Bos taurus myoglobin MYG; constructing a first Escherichia coli production host containing the first plasmid and the second plasmid; and producing the bovine myoglobin by culturing the first Escherichia coli production host. A composition useful as a meat flavor and/or an iron supplement includes the bovine myoglobin prepared in accordance with the method.

This application claims priority to US Provisional Patent Application No. 62/959,708, filed on Jan. 10, 2020, which is incorporated by reference for all purposes as if fully set forth herein.

### BACKGROUND OF THE INVENTION

### Field of the Invention

The present invention relates to a method for preparing bovine myoglobin using Escherichia coli and the use of bovine myoglobin as a meat flavor and an iron supplement.

### Discussion of the Related Art

Humans began eating meat from the beginning of their hunting lives, and meat was obtained primarily from the flesh (muscle tissue) of the hunted animals. As humankind developed, so did all industries, but there were some problems to be solved. Among them, the development of the livestock industry brought about various problems, including environmental problems. For example, environmental issues include the livestock industry, which accounts for about 15% of all human greenhouse gas emissions, half of which comes from 1.5 billion cattle raised around the world. Animal meat also requires 4-25 times more water and 6-17 times more land than the same amount of vegetation. In addition, slaughtering to obtain meat has become a problem in animal ethics as the awareness of the right to life of animals has recently increased. The more serious problem is that as the world population increase rapidly, there is a limit to the meat supply in the same way as it is today.

Meat-analogue is attracting attention as a major tool to solve vicious cycle of the inefficiency, anti-environmental and anti-health behind animal meat. Generally, meat-analogue means a food made from vegetarian ingredients, and sometimes without animal products such as dairy. Many meat-analogues are soy-based (e.g. tofu, tempeh) or gluten-based, but now may also be made from pea protein. The target market for meat-analogues includes vegetarians, vegans, non-vegetarians seeking to reduce their meat consumption, and people following religious dietary laws in Hinduism, Judaism, Islam, and Buddhism. According to a recent report, global meat-analogue market size was valued at $4,175 million in 2017, and is expected to reach $7,549 million by 2025. The rationale for the importance of meat-analogues often starts with feeding a global population projected to grow from 7.7 billion now to 9.8 billion in 2050 and 11.2 billion in 2100. Most of this growth will occur in Africa, followed by Asia.

Despite the importance of the meat-analogue development, meatless meat products on the market today are different in one important way. Meat-analogues are made from plants to give the same texture of food as meat. Most companies that produce meat-analogues choose to unique meat color by adding beet juice or other vegetable pigments to the meat-analogues, but they cannot provide meat like flavor.

Therefore, it is more necessary than anything else to develop a food additive that can provide and preserve the flavor of real meat as well as the color.

Meanwhile, iron (Fe) is a trace element that plays an essential role for oxygen transport in the body, and is an important constituent of hemoglobin, myoglobin, cytochrome, iron/sulfur protein and biomolecular structures. The total mean amount of iron in the body is about 3 to 4 g, 60 to 65% of which is bound to hemoglobin in circulating erythrocytes, and the remaining 30 to 35% is present as storage iron (ferritin). Iron is also present in the form of tissue iron and serum iron (transferrin), and furthermore, there is a small amount of iron in myoglobin of the muscles.

Iron is not synthesized in the body and thus must be acquired entirely through intake, and exists in two types, heme iron and non-heme iron. Heme iron is an iron complex having a moiety structurally identical to the heme of hemoglobin in the body, and non-heme iron is an iron complex not having a moiety structurally identical to the heme of hemoglobin. These two types of iron may be used as iron supplements (iron supplementary compound), and the bioavailability of heme iron is known to be much higher than that of non-heme iron. Also, the absorption of heme iron in the body is not affected by other dietary factors. Moreover, heme iron has the advantage of not causing various side effects (constipation, gastrointestinal disorders, etc.) that have been reported for non-heme iron.

Generally, heme iron is manufactured from blood of slaughtered animal, such as porcine blood. The heme iron is prepared from slaughterhouse blood by a manner in which hemoglobin is first separated from the slaughterhouse blood and then heme iron is isolated from the separated hemoglobin. The separation of heme iron from hemoglobin may be performed through a method of using an alcohol and an imidazole derivative (Lindroos, U.S. Pat. No. 4,431,581), a method of adding amino acids thereto (Ingberg, et. al., US Patent 5,008,388), a method of performing decomposition at a high temperature using a highly concentrated organic acid (Liu, et. al., J. Agric. Food Chem., 44, 2957, 1996), a method of using a protease, and the like.

Heme iron thus prepared by conventional method has many problems that are not present in non-heme iron, such as the risk of infection by animal-derived infection sources, livestock growth hormone contamination, and residual antibiotics. Therefore, it is necessary to develop a method of preparing heme iron not derived from animal blood.

### SUMMARY OF THE INVENTION

Accordingly, the present invention has been made keeping in mind the problems encountered in the related art and is intended to solve such problems.

In an aspect, a method for preparing a bovine myoglobin includes: constructing a first plasmid containing genes for heme biosynthesis pathway enzymes; constructing a second plasmid containing a gene for Bos taurus myoglobin MYG; constructing a first Escherichia coli production host containing the first plasmid and the second plasmid; and producing the bovine myoglobin by culturing the first Escherichia coli production host.

In another aspect, the heme biosynthesis pathway enzymes are an ALA synthase, a NADP-dependent malic enzyme, a dicarboxylic acid transporter and a ferrochelatase.

In another aspect, the bovine myoglobin consists of a globin having an amino acid sequence as set forth in SEQ ID NO: 1 and a heme having formula 1.

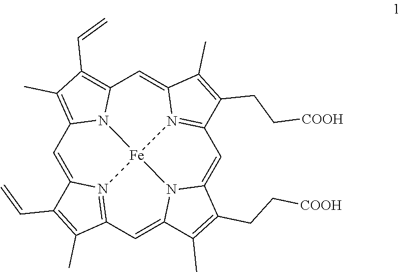

In another aspect, the first plasmid has a nucleotide sequence set forth in SEQ ID NO: 6.

In another aspect, the second plasmid has a nucleotide sequence set forth in SEQ ID NO: 8.

In another aspect, the ALA synthase is a Rhodobacter sphaeroides ALA synthase having a nucleotide sequence set forth in SEQ ID NO: 2, the NADP-dependent malic enzyme is an Escherichia coli NADP-dependent malic enzyme having a nucleotide sequence set forth in SEQ ID NO: 3, the dicarboxylic acid transporter is an Escherichia coli dicarboxylic acid transporter having a nucleotide sequence set forth in SEQ ID NO: 4, and the ferrochelatase is an Escherichia coli ferrochelatase having a nucleotide sequence set forth in SEQ ID NO: 5.

In another aspect, the method further includes: adjusting pH to 7 to 9 using succinic acid for the culturing the first Escherichia coli production host.

In an aspect, a method for preparing a bovine myoglobin includes: constructing a third plasmid containing genes for heme biosynthesis pathway enzymes; constructing a second Escherichia coli production host containing the third plasmid; and producing the bovine myoglobin by culturing the second Escherichia coli production host.

In another aspect, the heme biosynthesis pathway enzymes are an ALA synthase, a NADP-dependent malic enzyme, a dicarboxylic acid transporter and a ferrochelatase.

In another aspect, the bovine myoglobin consists of a globin having an amino acid sequence as set forth in SEQ ID NO: 1 and a heme having formula 1.

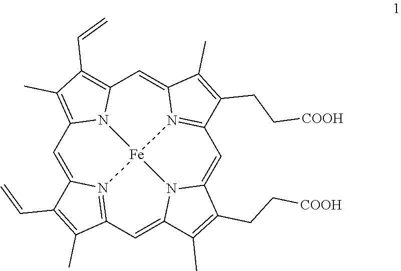

In another aspect, the third plasmid has a nucleotide sequence set forth in SEQ ID NO: 9.

In another aspect, the ALA synthase is a Rhodobacter sphaeroides ALA synthase having a nucleotide sequence set forth in SEQ ID NO: 2, the NADP-dependent malic enzyme is an Escherichia coli NADP-dependent malic enzyme having a nucleotide sequence set forth in SEQ ID NO: 3, the dicarboxylic acid transporter is an Escherichia coli dicarboxylic acid transporter having a nucleotide sequence set forth in SEQ ID NO: 4, and the ferrochelatase is an Escherichia coli ferrochelatase having a nucleotide sequence set forth in SEQ ID NO: 5.

In another aspect, the method further includes: adjusting pH to 7 to 9 using succinic acid for the culturing the second Escherichia coli production host.

In an aspect, a method for preparing a bovine myoglobin includes: constructing a second plasmid containing a gene for Bos taurus myoglobin MYG; constructing a third Escherichia coli production host containing the second plasmid; producing a globin by culturing the third Escherichia coli production host; producing a heme by microbial fermentation or chemical synthesis; and coupling of the globin and the heme to obtain the bovine myoglobin.

In another aspect, the second plasmid has a nucleotide sequence set forth in SEQ ID NO: 8.

In another aspect, the producing the heme includes: constructing a first plasmid containing genes for heme biosynthesis pathway enzymes; constructing a fourth Escherichia coli production host containing the first plasmid; and producing the heme by culturing the fourth Escherichia coli production host.

In another aspect, the first plasmid has a nucleotide sequence set forth in SEQ ID NO: 6.

In an aspect, a composition useful as a meat flavor and/or an iron supplement includes the bovine myoglobin prepared in accordance with the method.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

In the drawings:

FIG. 1 depicts a plasmid map of pLEX_HMDH.

FIG. 2 depicts a plasmid map of pBAD_BMYG.

FIG. 3 depicts a plasmid map of pLEX_BHMDH.

FIG. 4 is the result of SDS-PAGE analysis. Lane M: Protein marker, lane 1: Globin, lane 2: Example 8, lane 3: Example 9, lane 4: Example 10-4, and lane 5: Example 10-5.

FIG. 5 is the result of Native PAGE analysis. Lane 1: Globin, lane 2: Example 8, lane 3: Example 9, lane 4: Example 10-4, and lane 5: Example 10-5. Red arrow: Heme-globin complex.

FIG. 6 is the result of spectral analysis.

FIG. 7 is the result of fluorescence spectroscopy analysis.

### DETAILED DESCRIPTION OF THE ILLUSTRATED EMBODIMENTS

Reference will now be made in detail to embodiments of the present invention, example of which is illustrated in the accompanying drawings.

In order to accomplish the above objectives, the present inventors have, as the result of intensive study, developed a process of preparing bovine myoglobin, and a composition containing heme-globin complex above prepared. The composition may be usefully utilized as a meat flavor and an iron supplement, thus culminating in the present invention.

As used herein, the term “heme iron” refers to an iron complex comprising a moiety having the same structure as the heme of hemoglobin in the body, and the term “non-heme iron” refers to an iron complex not comprising a moiety having the same structure as the heme of hemoglobin.

The globin of the present invention includes variants thereof having at least 80%, 85%, 90%, 95%, 99%, or 99.5% identity to the amino acid sequence of SEQ ID NO: 1, but not limited thereto. The amino acid sequence identity is defined herein as the percentage of amino acid residues in a candidate sequence that are identical with the amino acid residues in the globin sequence, after aligning the sequence in the same reading frame and introducing gaps, if necessary, to achieve the maximum percent sequence identity, and not considering any conservative substitutions as part of the sequence identity.

To determine the percent identity of two amino acid sequences, the sequences are aligned for optimal comparison purposes (e.g., gaps may be introduced in the sequence of a first sequence). The amino acids at corresponding amino acid positions are then compared. When a position in the first sequence is occupied by the same amino acid as the corresponding position in the second sequence, then the molecules are identical at that position. The percent identity between the two sequences is a function of the number of identical positions shared by the sequences (i.e., % identity=# of identical positions/total # of position×100).

The composition containing the bovine myoglobin of the present invention may additionally include food-grade components, which is exemplified by sugars, salts, preservatives and additives, but not limited thereto. The composition containing the bovine myoglobin of the present invention can additionally include emulsifiers, suspending agents, and stabilizer, in addition to the above ingredients, but not limited thereto.

The composition containing the bovine myoglobin of the present invention can be added to meat-analogues as meat flavor. The meat-analogues are exemplified by vegetable meat, cultured meat (cell-cultured meat), and synthetic meat, but not limited thereto.

The composition containing the bovine myoglobin of the present invention can be added to foods as iron supplement. The foods are exemplified by cracker, cookie, snack foods, and beverage, but not limited thereto.

The amount added to the meat-analogues or foods of the bovine myoglobin of the present invention varies from the type of meat-analogues or foods. In general, the bovine myoglobin will be added to the meat-analogues or foods to deliver not more than 1% (w/w) bovine myoglobin.

In the process of preparing the bovine myoglobin by the processes of Escherichia coli fermentation, Escherichia coli HMDH_BMYG-d or Escherichia coli HMDH_BMYG-s is used as a production host.

In the process of biologically preparing the bovine myoglobin using production host Escherichia coli HMDH_BMYG-d or Escherichia coli HMDH_BMYG-s, the pH of the culture process is maintained in the range of 7 to 9, and preferably in the range of 8 to 9. Here, the pH is adjusted using succinic acid. When succinic acid is used to adjust the pH in this process, succinic acid is a substance used as a substrate in the biosynthesis of bovine myoglobin, which is advantageous for the high efficient production of the bovine myoglobin.

In the process of preparing the bovine myoglobin by the processes of in vitro coupling of separately manufactured globin and heme, Escherichia coli BMYG is used as a production host of globin.

In the process of preparing the bovine myoglobin by the processes of in vitro coupling of separately manufactured globin and heme, Escherichia coli HMDH is used as a production host of heme.

In the process of preparing the bovine myoglobin by the processes of in vitro coupling of separately manufactured globin and heme, the heme may be produced by chemical process.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### EXAMPLE 1

### Construction of the Plasmid pLEX_HMDH

An expression plasmid comprising four core enzymes of the heme biosynthesis pathway of the present invention was constructed by conventional subcloning genes encoding the Rhodobacter sphaeroides ALA synthase (HemA), Escherichia coli NADP-dependent malic enzyme (MaeB), Escherichia coli dicarboxylic acid transporter (DctA) and Escherichia coli ferrochelatase (HemH) into pLEX vector (Invitrogen). The nucleotide sequence of Rhodobacter sphaeroides ALA synthase is presented by SEQ ID NO: 2; the nucleotide sequence of Escherichia coli NADP-dependent malic enzyme is presented by SEQ ID NO: 3; the nucleotide sequence of Escherichia coli dicarboxylic acid transporter is presented by SEQ ID NO: 4; and the nucleotide sequence of Escherichia coli ferrochelatase is presented by SEQ ID NO: 5.

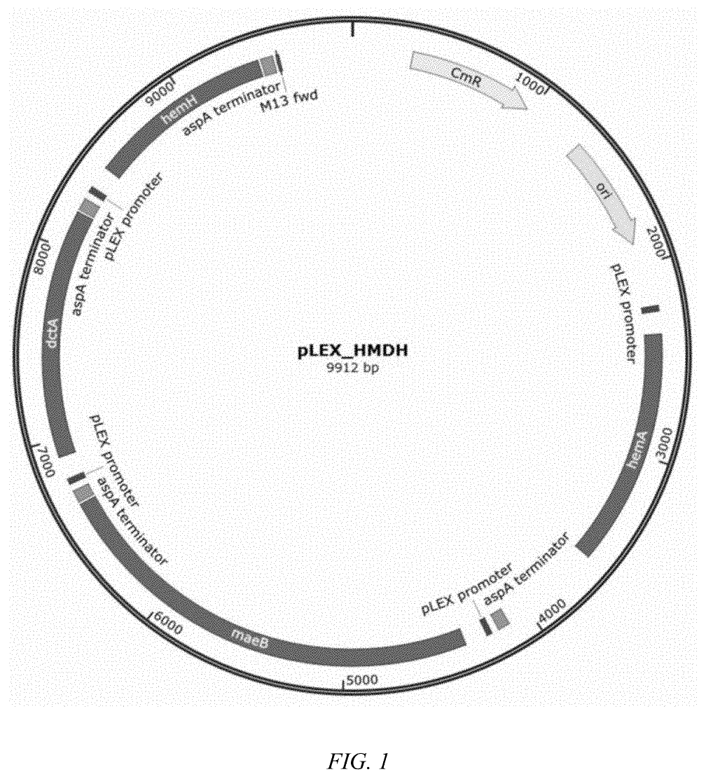

The resulting plasmid was named as pLEX_HMDH. In the plasmid pLEX_HMDH, each inserted gene has individual P_{L} promoter and aspA transcriptional terminator in front of and behind each gene (FIG. 1).

The nucleotide sequence of pLEX_HMDH is presented by SEQ ID NO: 6.

### EXAMPLE 2

### Construction of the Plasmid pBAD_BMYG

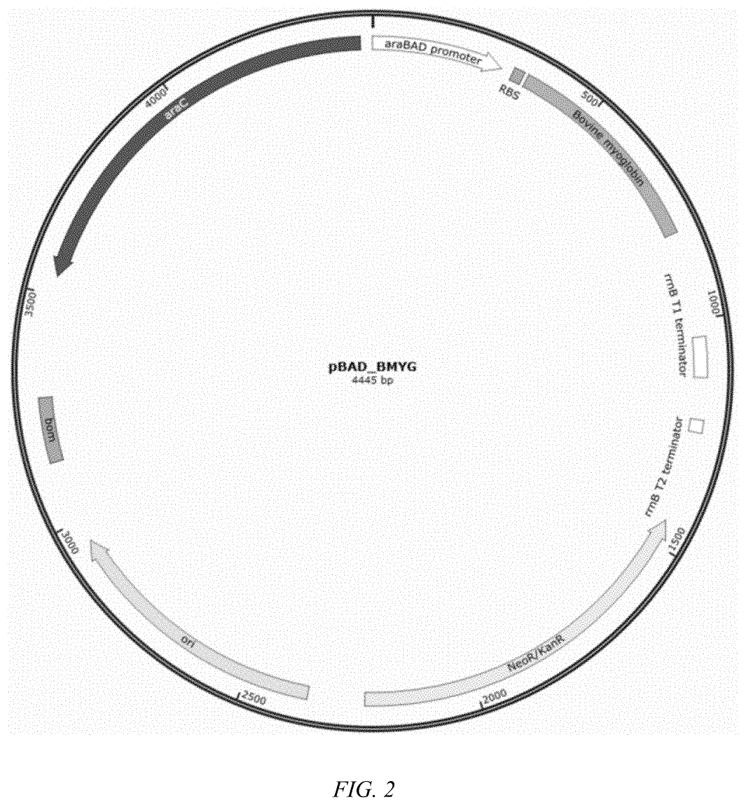

The coding sequence for the Bos taurus myoglobin MYG was codon-optimized for expression in Escherichia coli, chemically synthesized and cloned into pBAD vector (Invitrogen), resulting in the plasmid pBAD_BMYG (FIG. 2).

The plasmid pBAD_BMYG contains the coding sequence for the globin protein of bovine myoglobin.

The codon-optimized nucleotide sequence of Bos taurus myoglobin MYG is presented by SEQ ID NO: 7 and the nucleotide sequence of pBAD_BMYG is presented by SEQ ID NO: 8.

### EXAMPLE 3

### Construction of the Plasmid pLEX_BHMDH

An expression plasmid comprising four core enzymes of the heme biosynthesis pathway and the Bos taurus myoglobin MYG of the present invention was constructed by conventional subcloning genes encoding the Rhodobacter sphaeroides ALA synthase (HemA), Escherichia coli NADP-dependent malic enzyme (MaeB), Escherichia coli dicarboxylic acid transporter (DctA), Escherichia coli ferrochelatase (HemH) and the codon-optimized nucleotide sequence of Bos taurus myoglobin MYG into pLEX vector (Invitrogen). The nucleotide sequence of Rhodobacter sphaeroides ALA synthase is presented by SEQ ID NO: 2; the nucleotide sequence of Escherichia coli NADP-dependent malic enzyme is presented by SEQ ID NO: 3; the nucleotide sequence of Escherichia coli dicarboxylic acid transporter is presented by SEQ ID NO: 4; the nucleotide sequence of Escherichia coli ferrochelatase is presented by SEQ ID NO: 5; and the codon-optimized nucleotide sequence of Bos taurus myoglobin MYG is presented by SEQ ID NO: 7.

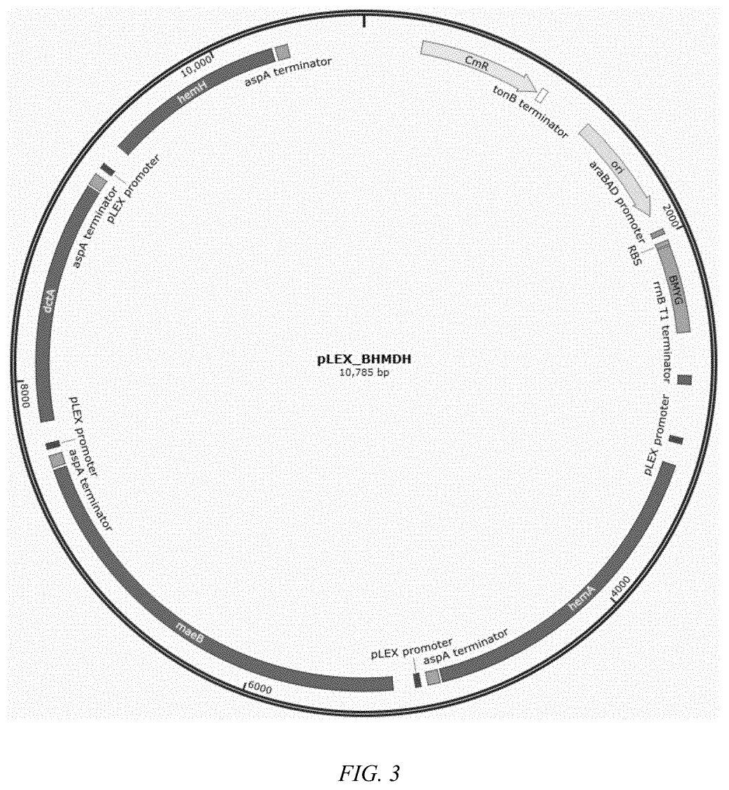

The resulting plasmid was named as pLEX_BHMDH. In the plasmid pLEX_BHMDH, each inserted gene encoding the heme synthetic enzymes has separate PL promoter and aspA transcriptional terminator in front of and behind each gene and the inserted gene encoding the Bos taurus myoglobin MYG has araBAD promoter and rrnB T1 terminator (FIG. 3). The nucleotide sequence of pLEX BHMDH is presented by SEQ ID NO: 9.

### EXAMPLE 4

### Construction of the Production Host for Heme

Escherichia coli K-12 DH10B cell transformed with plasmid pLEX HMDH was used as a production host for heme of the present invention. The constructed production host was named as Escherichia coli HMDH.

As the source inoculum for the production of heme, frozen cell banks for the production host Escherichia coli HMDH in 25% glycerol (v/v) were maintained at −80° C.

### EXAMPLE 5

### Construction of the Production Host for Globin

Escherichia coli K-12 DH10B cell transformed with plasmid pBAD_BMYG was used as a production host for globin of the present invention. The constructed production host was named as Escherichia coli BMYG.

As the source inoculum for the production of globin, frozen cell banks for the production host Escherichia coli BMYG in 25% glycerol (v/v) were maintained at −80° C.

### EXAMPLE 6

### Construction of the Production Host for Bovine Myoglobin using Plasmids pLEX_HMDH and pBAD_BMYG

Escherichia coli K-12 DH10B cell transformed with two expression constructs (pLEX_HMDH and pBAD_BMYG) was used as a production host for bovine myoglobin of the present invention. The constructed production host was named as Escherichia coli HMDH_BMYG-d.

As the source inoculum for the production of bovine myoglobin, frozen cell banks for the production host Escherichia coli HMDH_BMYG-d in 25% glycerol (v/v) were maintained at −80° C.

### Example 7

### Construction of the Production Host for Bovine Myoglobin using Plasmid pLEX_BHMDH

Escherichia coli K-12 DH10B cell transformed with plasmid pLEX_BHMDH was used as a production host for bovine myoglobin of the present invention. The constructed production host was named as Escherichia coli HMDH_BMYG-s.

As the source inoculum for the production of bovine myoglobin, frozen cell banks for the production host Escherichia coli HMDH_BMYG-s in 25% glycerol (v/v) were maintained at −80° C.

### EXAMPLE 8

### Production of Bovine Myoglobin using Production Host Escherichia coli HMDH_BMYG-d

bovine myoglobin was produced by microbial fermentation using the Escherichia coli HMDH BMYG-d (production host).

10 ml of a LB (Luria-Bertani) medium (10 g/L peptone, 5 g/L yeast extract, and 10 g/L NaCl) containing 50 μg/ml chloramphenicol and 50 μg/ml kanamycin was added in a 50 ml conical tube, and production host was seeded therein and then cultured overnight at 37° C. and 200 rpm using a rotary shaking incubator. Next, 1 ml of the culture broth obtained after overnight culture was seeded in 250 ml Erlenmeyer flasks added with 60 ml of an S medium (10 g/L peptone, 5 g/L yeast extract, 5 g/L KH₂PO₄, 10 g/L succinate, 2 g/L glycine, and 10 mg/L FeCl₂.4H₂O) containing 50 μg/ml chloramphenicol and 50 μg/ml kanamycin, and was then cultured at 37° C. and 200 rpm for 4 hr. After culturing for 4 hr, the resultant culture solution was inoculated in 5 L fermenter containing 3 L of an S medium containing 50 μg/ml chloramphenicol and 50 μg/ml kanamycin. The culture solution in the fermenter was cultured at 37° C., 0.5 vvm aeration and 200 rpm until culture reaches OD₆₀₀ of 0.5. When the culture reaches OD_{600=0.5,} L-arabinose (final concentration=0.2%) solution was added in the culture solution. And the culture solution in the fermenter was cultured for additional 72 hr (37° C., 0.5 vvm, 200 rpm). During the fermentation process, the pH is maintained at 8-9 and the pH adjustment is controlled by using succinic acid feeding. Using succinic acid to control pH can provide the advantage that succinic acid is a substance used as a substrate in the biosynthesis of heme, which is ultimately advantageous for the high efficient production of composition. After fermentation, the resulting cells were recovered by centrifugation at 4,500×g at 4° C. for 15 minutes.

The cell pellet obtained from centrifugation of fermentation broth was lysed by sonication. Specifically, the cells were resuspended in 50 ml of 20 mM Tris-HCl buffer (pH 8.0). The cells in this cell suspension were disrupted by sonication as follows; sonication was performed for 20 seconds to disrupt cells and stopped to take a break for 5 seconds, which was repeated for 20 minutes. The obtained whole cell lysate was centrifuged again (25,000×g, 10 minutes) to separate precipitate and supernatant.

Ammonium sulfate precipitation was performed with the above resultant supernatant to concentrate the prepared bovine myoglobin. More precisely, the resultant supernatant was adjusted to 40% saturation with solid ammonium sulfate and stirred for 2 hr. Precipitated material was removed by centrifugation at 25,000×g at 4° C. for 15 min, and the supernatant made to 70% saturation with solid ammonium sulfate. This solution was stirred for 2 hr, prior to recovery of the precipitate by centrifugation at 25,000×g at 4° C. for 30 min. Precipitated bovine myoglobin was resuspended in 5 ml of 50 mM Tris-HCl buffer (pH 8.0). To remove the excessive ammonium sulfate, Sephadex G-25 (GE Healthcare) was used as the desalting resin. The column was packed with the Sephadex G-25 by 2.6×10 cm and at this time the total packed bed volume was approximately 50 ml. The column was equilibrated with the 50 mM Tris-HCl buffer (pH 8.0) before sample loading. Then, the sample containing the bovine myoglobin was loaded onto the column. Then the column was flowed with the 50 mM Tris-HCl buffer (pH 8.0) and collected the fraction with peak of the protein.

The desalted fraction was filtered with 0.2-μm filter, followed by anion-exchange chromatography. HiTrap Q FF anion-exchange chromatography column was packed with the Q Sepharose fast flow anion exchange resin (GE Healthcare), and at this time the total packed bed volume was approximately 5 ml. The column was equilibrated with the adsorption buffer (50 mM Tris-HCl, pH 8.0) before sample loading. Then, the sample containing the bovine myoglobin was loaded onto the column, followed by washing with 25 ml (5 column volumes) of the adsorption buffer. The bovine myoglobin was eluted by using 50 mM of Tris-HCl solution (pH 8.0) containing 0.1 M sodium chloride. To remove sodium chloride used for the elution of the bovine myoglobin, the eluent containing the bovine myoglobin was dialyzed against 50 mM of Tris-HCl solution (pH 8.0) at 4° C. by centrifugation (4,500 rpm, 10 minutes) using AMICON Ultra-15 3K centrifugal filter (Millipore). At the same time, dialyzed bovine myoglobin was concentrated and stored at −20° C. until use.

### EXAMPLE 9

### Production of Bovine Myoglobin using Production Host Escherichia coli HMDH_BMYG-s

bovine myoglobin was produced by microbial fermentation using the Escherichia coli HMDH_BMYG-s (production host).

10 ml of a LB (Luria-Bertani) medium (10 g/L peptone, 5 g/L yeast extract, and 10 g/L NaCl) containing 50 μg/ml chloramphenicol was added in a 50 ml conical tube, and production host was seeded therein and then cultured overnight at 37° C. and 200 rpm using a rotary shaking incubator. Next, 1 ml of the culture broth obtained after overnight culture was seeded in 250 ml Erlenmeyer flasks added with 60 ml of an S medium (10 g/L peptone, 5 g/L yeast extract, 5 g/L KH₂PO4, 10 g/L succinate, 2 g/L glycine, and 10 mg/L FeCl_{2.4}H20) containing 50 μg/ml chloramphenicol, and was then cultured at 37° C. and 200 rpm for 4 hr. After culturing for 4 hr, the resultant culture solution was inoculated in 5 L fermenter containing 3 L of an S medium containing 50 μg/ml chloramphenicol. The culture solution in the fermenter was cultured at 37° C., 0.5 vvm aeration and 200 rpm until culture reaches OD₆₀₀ of 0.5. When the culture reaches OD₆₀₀=0.5, L-arabinose (final concentration=0.2%) solution was added in the culture solution. And the culture solution in the fermenter was cultured for additional 72 hr (37° C., 0.5 vvm, 200 rpm). During the fermentation process, the pH is maintained at 8-9 and the pH adjustment is controlled by using succinic acid feeding. Using succinic acid to control pH can provide the advantage that succinic acid is a substance used as a substrate in the biosynthesis of heme, which is ultimately advantageous for the production of high-efficiency composition. After fermentation, the resulting cells were recovered by centrifugation at 4,500×g at 4° C. for 15 minutes.

The cell pellet obtained from centrifugation of fermentation broth was lysed by sonication. Specifically, the cells were resuspended in 50 ml of 20 mM Tris-HCl buffer (pH 8.0). The cells in this cell suspension were disrupted by sonication as follows; sonication was performed for 20 seconds to disrupt cells and stopped to take a break for 5 seconds, which was repeated for 20 minutes. The obtained whole cell lysate was centrifuged again (25,000×g, 10 minutes) to separate precipitate and supernatant.

Ammonium sulfate precipitation was performed with the above resultant supernatant to concentrate the prepared bovine myoglobin. More precisely, the resultant supernatant was adjusted to 40% saturation with solid ammonium sulfate and stirred for 2 hr. Precipitated material was removed by centrifugation at 25,000×g at 4° C. for 15 min, and the supernatant made to 70% saturation with solid ammonium sulfate. This solution was stirred for 2 hr, prior to recovery of the precipitate by centrifugation at 25,000×g at 4° C. for 30 min. Precipitated bovine myoglobin was resuspended in 5 ml of 50 mM Tris-HCl buffer (pH 8.0). To remove the excessive ammonium sulfate, Sephadex G-25 (GE Healthcare) was used as the desalting resin. The column was packed with the Sephadex G-25 by 2.6×10 cm and at this time the total packed bed volume was approximately 50 ml. The column was equilibrated with the 50 mM Tris-HCl buffer (pH 8.0) before sample loading. Then, the sample containing the bovine myoglobin was loaded onto the column. Then the column was flowed with the 50 mM Tris-HCl buffer (pH 8.0) and collected the fraction with peak of the protein.

The desalted fraction was filtered with 0.2-μm filter, followed by anion-exchange chromatography. HiTrap Q FF anion-exchange chromatography column was packed with the Q Sepharose fast flow anion exchange resin (GE Healthcare), and at this time the total packed bed volume was approximately 5 ml. The column was equilibrated with the adsorption buffer (50 mM Tris-HCl, pH 8.0) before sample loading. Then, the sample containing the bovine myoglobin was loaded onto the column, followed by washing with 25 ml (5 column volumes) of the adsorption buffer. The bovine myoglobin was eluted by using 50 mM of Tris-HCl solution (pH 8.0) containing 0.1 M sodium chloride. To remove sodium chloride used for the elution of the bovine myoglobin, the eluent containing the bovine myoglobin was dialyzed against 50 mM of Tris-HCl solution (pH 8.0) at 4° C. by centrifugation (4,500 rpm, 10 minutes) using AMICON Ultra-15 3K centrifugal filter (Millipore). At the same time, dialyzed bovine myoglobin was concentrated and stored at −20° C. until use.

### EXAMPLE 10

### Production of Bovine Myoglobin by In Vitro Coupling of Separately Manufactured Globin and Heme

### EXAMPLE 10-1

### Production of Globin

Globin was produced by microbial fermentation using the Escherichia coli BMYG (production host).

10 ml of a LB (Luria-Bertani) medium (10 g/L peptone, 5 g/L yeast extract, and 10 g/L NaCl) containing 50 μg/ml kanamycin was added in a 50 ml conical tube, and production host was seeded therein and then cultured overnight at 37° C. and 200 rpm using a rotary shaking incubator. Next, 5 ml of the culture broth obtained after overnight culture was seeded in 2 L Erlenmeyer flask added with 500 ml of a LB medium containing 50 μg/ml kanamycin, and was then incubated at 37° C. and 200 rpm until culture reaches OD₆₀₀ of 0.5. When the culture reaches OD₆₀₀=0.5, L-arabinose (final concentration=0.2%) solution was added in 2 L Erlenmeyer flask containing 50 μg/ml kanamycin. The culture solution in the 2 L Erlenmeyer flask was cultured overnight at 25° C. and 150 rpm using a rotary shaking incubator. After incubation, the resulting cells were recovered by centrifugation at 4,500×g at 4° C. for 15 minutes.

The cell pellet obtained from centrifugation of culture broth was lysed by sonication. Specifically, the cells were resuspended in 50 ml of 20 mM Tris-HCl buffer (pH 8.0). The cells in this cell suspension were disrupted by sonication as follows; sonication was performed for 20 seconds to disrupt cells and stopped to take a break for 5 seconds, which was repeated for 20 minutes. The obtained whole cell lysate was centrifuged again (25,000×g, 10 minutes) to separate precipitate and supernatant.

Ammonium sulfate precipitation was performed with the above resultant supernatant to concentrate the prepared globin. More precisely, the resultant supernatant was adjusted to 40% saturation with solid ammonium sulfate and stirred for 2 hr. Precipitated material was removed by centrifugation at 25,000×g at 4° C. for 15 min, and the supernatant made to 70% saturation with solid ammonium sulfate. This solution was stirred for 2 hr, prior to recovery of the precipitate by centrifugation at 25,000×g at 4° C. for 30 min. Precipitated the globin was resuspended in 5 ml of 50 mM Tris-HCl buffer (pH 8.0). To remove the excessive ammonium sulfate, Sephadex G-25 (GE Healthcare) was used as the desalting resin. The column was packed with the Sephadex G-25 by 2.6×10 cm and at this time the total packed bed volume was approximately 50 ml. The column was equilibrated with the 50 mM Tris-HCl buffer (pH 8.0) before sample loading. Then, the sample containing the globin was loaded onto the column. Then the column was flowed with the 50 mM Tris-HCl buffer (pH 8.0) and collected the fraction with peak of the protein.

The desalted fraction was filtered with 0.2-μm filter, followed by anion-exchange chromatography. HiTrap Q FF anion-exchange chromatography column was packed with the Q Sepharose fast flow anion exchange resin (GE Healthcare), and at this time the total packed bed volume was approximately 5 ml. The column was equilibrated with the adsorption buffer (50 mM Tris-HCl, pH 8.0) before sample loading. Then, the sample containing the globin was loaded onto the column, followed by washing with 25 ml (5 column volumes) of the adsorption buffer. The globin was eluted by using 50 mM of Tris-HCl solution (pH 8.0) containing 0.1 M sodium chloride. To remove sodium chloride used for the elution of the globin, the eluent containing the globin was dialyzed against 50 mM of Tris-HCl solution (pH 8.0) at 4° C. by centrifugation (4,500 rpm, 10 minutes) using AMICON Ultra-15 3K centrifugal filter (Millipore). At the same time, dialyzed globin was concentrated and stored at −20° C. until use.

### EXAMPLE 10-2

### Production of Heme by Biological Process

Heme was produced by microbial fermentation using the Escherichia coli HMDH (production host).

10 ml of a LB (Luria-Bertani) medium (10 g/L peptone, 5 g/L yeast extract, and 10 g/L NaCl) containing 50 μg/ml chloramphenicol was added in a 50 ml conical tube, and production host was seeded therein and then cultured overnight at 37° C. and 200 rpm using a rotary shaking incubator. Next, 1 ml of the culture broth obtained after overnight culture was seeded in 250 ml Erlenmeyer flask added with 50 ml of an S medium (10 g/L peptone, 5 g/L yeast extract, 5 g/L KH₂PO₄, 10 g/L succinate, 2 g/L glycine, and 10 mg/L FeCl₂.4H₂O) containing 50 μg/ml chloramphenicol, and was then cultured at 37° C. and 200 rpm for 4 hr. After culturing for 4 hr, the resultant culture solution was inoculated in 10 L fermenter containing 5 L of an S medium containing 50 μg/ml chloramphenicol. The culture solution in the fermenter was cultured for 72 hr (37° C., 0.5 vvm aeration, 200 rpm). During the fermentation process, the pH is maintained at 8-9 and the pH adjustment is controlled by using succinic acid feeding. Using succinic acid to control pH can provide the advantage that succinic acid is a substance used as a substrate in the biosynthesis of heme, which is ultimately advantageous for the production of high-efficiency heme. After fermentation, the resulting cells were recovered by centrifugation at 3,000×g at 4° C. for 15 minutes.

The recovered cells were washed two times by suspending the same in PBS (Phosphate Buffered Saline) and then performing centrifugation. The finally recovered cells were naturally dried for about 30 minutes and then weighed. Typically, it was possible to recover 40 to 50 g of cells from 5 L of a culture broth. The recovered cells were added with cold acid-acetone and thus heme was extracted. Here, the cold acid-acetone that was used was prepared by mixing 998 ml of acetone at −20° C. with 2 ml of hydrochloric acid (HCl). The addition of the cold acid-acetone was conducted by a manner in which 1 L of cold acid-acetone was added to the cells recovered from 5 L of the culture broth. The extraction of heme using acid-acetone was performed at 4° C. for 5 days. The solution obtained through heme extraction for 5 days was passed through a celite-packed column to thus recover acetone containing heme. The acetone containing heme thus obtained was concentrated using a rotary evaporator. Here, concentration was performed until the volume was reduced from 1 L to 30 ml. The solution thus obtained was added with a 10-fold volume of methylene chloride, mixed thoroughly and then allowed to stand until layers were separated. After separation of the layers, the lower layer was recovered and concentrated using a rotary evaporator. Here, concentration was performed until the volume became 30 ml. After concentration, a NaOH aqueous solution was added in an amount of 2.1 equivalents based on the equivalents of heme contained in the concentrate, mixed thoroughly and then allowed to stand until layers were separated. After separation of the layers, the upper layer was recovered and stored at 4° C. until use. Or freeze-dried the upper layer and dissolve it in water when used.

### EXAMPLE 10-3

### Production of Heme by Chemical Synthesis

Heme was produced by chemical synthesis process that coordinates iron ion (Fe²+) into protoporphyrin IX. Protoporphyrin IX (PPIX, 10 g, 17.8 mmol) was dissolved in tetrahydrofuran (150 ml), slowly added with FeCl_{2.4}H₂O (14.4 g, 53.3 mmol), and refluxed at 85° C. for 4 hr. After termination of the reaction, the organic solvent was removed through vacuum distillation. Next, the reaction mixture was added with a NaOH aqueous solution and was thus dissolved therein, the resulting solution was filtered through a column packed with celite® 545, and the filtrate thus obtained was neutralized, thereby yielding chemical synthesized heme of free acid form (10.8 g, 99%). A solution of NaOH (630 mg, 15.9 mmol) dissolved in distilled water (15 ml) was added to heme of free acid form (5 g, 8.11 mmol) obtained in above and subjected to chlorination with stirring at room temperature for 30 minutes. After termination of the reaction, the reaction mixture was frozen at −80° C. and then freeze-dried and thus dewatered, thereby yielding chemical synthesized heme of salt form (5.25 g, 98%).

### EXAMPLE 10-4

### In Vitro Coupling of Separately Manufactured Globin and Biological Heme

In vitro coupling of the globin obtained from Example 10-1 and the heme obtained from Example 10-2 was performed. More precisely, 10 ml of 100 μM globin solution and 10 ml of 1 mM heme solution (molar ratio=1:10) were added to the 50 ml conical tube, and then slightly vortexed to react at room temperature for 30 minutes.

After reaction, heme-globin complex solution was filtered with 0.2-μm filter, followed by anion-exchange chromatography. HiTrap Q FF anion-exchange chromatography column was packed with the Q Sepharose fast flow anion exchange resin (GE Healthcare), and at this time the total packed bed volume was approximately 5 ml. The column was equilibrated with the adsorption buffer (50 mM Tris-HCl, pH 8.0) before sample loading. Then, the sample containing the heme-globin complex was loaded onto the column, followed by washing with 25 ml (5 column volume) of the adsorption buffer. The heme-globin complex was eluted by using 50 mM of Tris-HCl solution (pH 8.0) containing 0.1 M sodium chloride. To remove sodium chloride used for the elution of the heme-globin complex, the eluent containing the heme-globin complex was dialyzed against 50 mM of Tris-HCl solution (pH 8.0) at 4° C. by centrifugation (4,500 rpm, 10 minutes) using AMICON Ultra-15 3K centrifugal filter (Millipore). At the same time, dialyzed heme-globin complex was concentrated and stored at −20° C. until use.

### EXAMPLE 10-5

### In Vitro Coupling of Separately Manufactured Globin and Chemically Synthesized Heme

In vitro coupling of the globin obtained from Example 10-1 and the heme obtained from Example 10-3 was performed. More precisely, 10 ml of 100 μM globin solution and 10 ml of 1 mM heme solution (molar ratio=1:10) were added to the 50 ml conical tube, and then slightly vortexed to react at room temperature for 30 minutes.

After reaction, heme-globin complex solution was filtered with 0.2-μm filter, followed by anion-exchange chromatography. HiTrap Q FF anion-exchange chromatography column was packed with the Q Sepharose fast flow anion exchange resin (GE Healthcare), and at this time the total packed bed volume was approximately 5 ml. The column was equilibrated with the adsorption buffer (50 mM Tris-HCl, pH 8.0) before sample loading. Then, the sample containing the heme-globin complex was loaded onto the column, followed by washing with 25 ml (5 column volume) of the adsorption buffer. The heme-globin complex was eluted by using 50 mM of Tris-HCl solution (pH 8.0) containing 0.1 M sodium chloride. To remove sodium chloride used for the elution of the heme-globin complex, the eluent containing the heme-globin complex was dialyzed against 50 mM of Tris-HCl solution (pH 8.0) at 4° C. by centrifugation (4,500 rpm, 10 minutes) using AMICON Ultra-15 3K centrifugal filter (Millipore). At the same time, dialyzed heme-globin complex was concentrated and stored at −20° C. until use.

### EXAMPLE 11

### Preparation of Bovine Myoglobin as Liquid Formulation

The solutions containing the bovine myoglobin obtained through the processes disclosed in Examples 8-10 were subjected to buffer exchange using sodium chloride and sodium ascorbate buffer, and then be adjusted in the final concentration to be 1 mg/ml or 10 mg/ml. The concentration adjusted solution was filtered using a 0.2-μm filter and frozen to prepare the composition as liquid formulation.

### EXAMPLE 12

### Preparation of Bovine Myoglobin as Freeze-Dried Formulation

It is known that proteins are relatively unstable in aqueous state and undergo chemical and physical degradation resulting in a loss of biological activity during processing and storage. Freeze-drying (also known as lyophilisation) is a method for preserving proteins for storage.

The concentration adjusted solution prepared Example 11 was freeze-dried to prepare the composition as freeze-dried formulation.

A description of the freeze-drying process is given in the following:
(A)Filtrating the concentration-adjusted solution using a 0.2-μm filter.
(B) Placing the bottle containing the filtered solution on a stainless steel tray.
(C) Loading the tray into the freeze dryer and lyophilizing the solution using the following freeze drying cycle:
   (C-1) Equilibrating at 4° C. for about 20 minutes.
   (C-2) Bringing the shelf temperature at -40° C. and maintaining for 12 hours.
   (C-3) Bringing the condenser temperature at -50° C.
   (C-4) Applying vacuum to the chamber.
   (C-5) When the vacuum reaches a value of 1,500 mtorr, raising shelf temperature up to −20° C. and maintaining for 16 hours.
   (C-6) Raising the shelf temperature up to 20° C. in the manner of increase of 10° C. per 1 hour and maintaining for 4 hours.
   (C-7) Breaking the vacuum.
(D) Stoppering and sealing the stoppered vials with the appropriate flip-off caps.

The freeze-dried formulation was stored at 4° C.

### EXAMPLE 13

### Identification of Bovine Myoglobin

In order to identify the bovine myoglobin obtained from Example 8, Example 9, Example 10-4 and Example 10-5, electrophoresis analysis (SDS-PAGE analysis and native PAGE analysis), spectral analysis and fluorescence spectroscopy analysis were performed. In case with the freeze-dried composition, prior to analysis, the freeze-dried composition was reconstituted using distilled water. Among electrophoresis, SDS-PAGE for confirming the size of globin was performed using 15% gel and native PAGE for migration shift of heme-globin complex was performed using 10% gel under non-denaturing and non-reducing condition. Spectral analysis was performed using a micro plate reader (Tecan, Infinite M200 PRO) and fluorescence spectroscopy analysis was performed using a fluorescence quenching method. Briefly describing the measurement of absorbance for spectra analysis, 100 pi of each samples was added into the wells of a transparent 96 well plate. And then the absorbance was measured from 280 nm to 500 nm using a micro plate reader. Fluorescence quenching is a technique used to study molecular interactions and is an easy method for the observation of ligand-protein binding such as heme-globin complex (Principles of Fluorescence Spectroscopy. 277-330). Excitation wavelength was 280 nm and emission wavelength was measured between 300 nm and 500 nm.

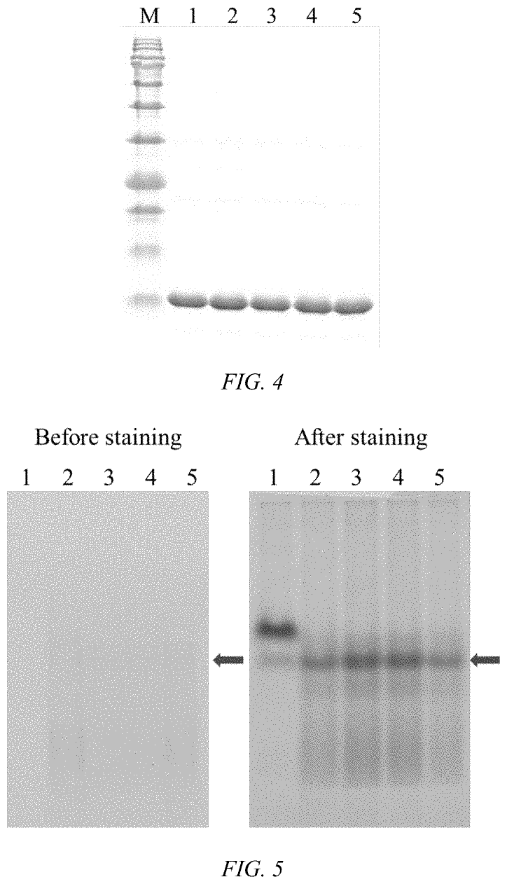

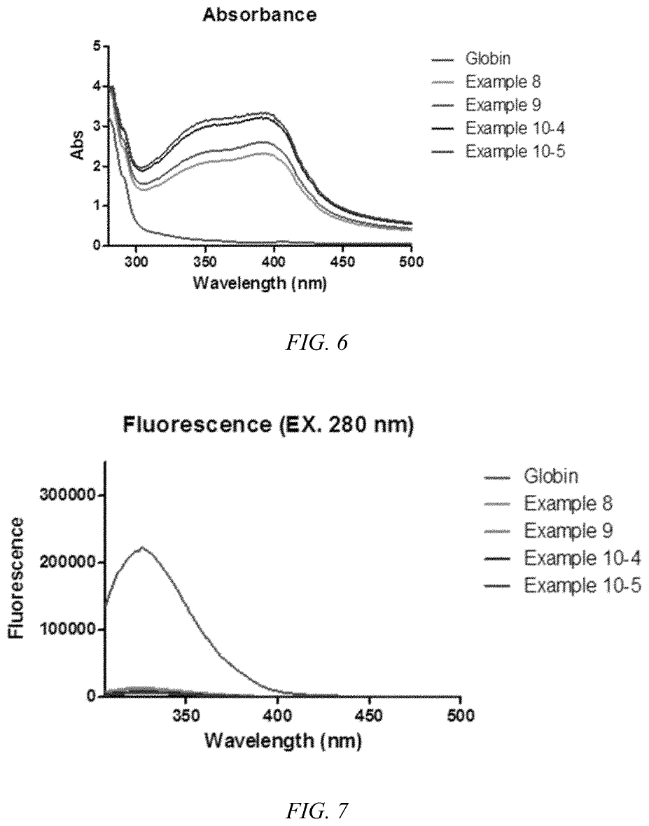

The Mr of globin and heme-globin complex was estimated by SDS-PAGE as approximately 13 kDa (FIG. 4). On the other hand, through the native PAGE analysis under native condition, band migration shift was shown between globin and heme-globin complex due to the difference of charge-to-mass ratio, physical shape and size of protein (FIG. 5). Also, the band was detected as brown band before gel staining (FIG. 5). Spectral analysis showed that the heme-globin complex had wide peaks from approximately 350 nm to 400 nm, while the maximum absorption wavelength of globin was at 280 nm (FIG. 6). Fluorescence spectroscopy analysis showed that maximum emission wavelength of globin was at 320 nm, while the fluorescence quenching was occurred in all samples of heme-globin complex, which is characteristic of bovine myoglobin (FIG. 7).

Based on above results, we concluded that bovine myoglobin was successfully manufactured.

### EXAMPLE 14

### Application of Bovine Myoglobin to Meat-Analogue as Flavor

Meat-analogue was prepared as follows. A dry mixture of the plant protein was added through a hopper into the extruder barrel and water is separately injected at room temperature. The extruder barrel is heated to a temperature between 80-150° C. The pressure on the front plate is between 10 to 20 bar. Also, oil is injected within this temperature range. The cooling die is cooling the product to an exit temperature of 70° C. The product was made on a twin screw extruder from the following materials:

TABLE 1
Composition of meat-analogue 1

| Ingredient | % (w/w) |
|---|---|
| Water | 63 |
| Soy protein concentrate | 32 |
| Plant oil | 4.2 |
| Flavoring by the composition containing | 0.8 |
| bovine myoglobin | |
| Total | 100 |

TABLE 2
Composition of meat-analogue 2

| Ingredient | % (w/w) |
|---|---|
| Water | 63 |
| Soy protein concentrate | 32 |
| Plant oil | 4.2 |
| Flavoring by beet juice extract | 0.8 |
| Total | 100 |

The resulted meat-analogue 1 and meat-analogue 2 have the appearance and texture of the meat. Meanwhile, 100 randomly chosen consumers compared the taste (flavor) of two meat-analogues. As a result, we found that meat-analogue 1 tasted better than meat-analogue 2 (78/100=78%).

### EXAMPLE 15

### Application of Bovine Myoglobin as an Iron Supplement

The composition containing bovine myoglobin prepared according to the present invention was administered to iron-deficiency-anemia-induced animals, whereby the effectiveness of the composition containing bovine myoglobin on alleviating anemia was evaluated.

Specifically, after 2 weeks acclimation period, thirty 9-week old Sprague-Dawley rats (female) were divided into 3 groups of 10 rats per group, among which one group was fed with normal feed in an amount of 10% of body weight daily for one month (Group 1; control), and the remaining two groups were fed with iron-deficient feed in an amount of 10% of body weight daily for 6 weeks to induce iron-deficiency anemia (Group 2 and Group 3). After 6 weeks of feeding, it was confirmed that iron-deficiency anemia was induced in the individual rat belonging to Group 2 and Group 3. Then, one of the anemia-induced groups was orally administered once a day with saline alone (Group 2), and the other anemia-induced group was orally administered once a day with solution containing bovine myoglobin (0.1 mg Fe/500 μ1 solution, Group 3). The administration continued for 5 weeks. During 5 weeks of administration to Group 2 and Group 3, Group 1 was continuously fed with normal feed, and Group 2 and Group 3 were fed with iron-deficient feed. The occurrence of abnormal symptoms was monitored during the administration period and there were no abnormal symptoms in any animals during the 5 weeks of administration period. After 5 weeks of administration, blood was collected, and whether anemia was alleviated was evaluated. The analysis results of blood collection are shown below.

TABLE 3
Analysis results of blood collection

| | | Weight of | | |
|---|---|---|---|---|
| | | rats on | | |
| | | 5 weeks after | Blood test | |
| Group | | administration | Hemoglobin | Hematocrit |
| No. | Treatment | [g] | content [g/dl] | [%] |
| Group | Normal feed | 334.3 ± 2.7 | 15.05 ± 0.9 | 35.9 ± 0.9 |
| 1 | | | | |
| Group | Iron-deficient | 299.7 ± 1.4 | 10.3 ± 0.7 | 22.2 ± 1.2 |
| 2 | feed + saline | | | |
| Group | Iron-deficient | 318.9 ± 1.1 | 14.0 ± 0.7 | 35.8 ± 1.9 |
| 3 | feed + heme- | | | |
| | globin complex | | | |

As indicated by the above results, the composition containing bovine myoglobin of the present invention can be concluded to be effective at alleviating iron-deficiency anemia and is thus efficient material as an iron supplementary source.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalents.

1. A method for preparing a bovine myoglobin comprising:
constructing a first plasmid containing genes for heme biosynthesis pathway enzymes;
constructing a second plasmid containing a gene for Bos taurus myoglobin MYG;
constructing a first Escherichia coli production host containing the first plasmid and the second plasmid; and
producing the bovine myoglobin by culturing the first Escherichia coli production host.

2. The method of claim 1, wherein the heme biosynthesis pathway enzymes are an ALA synthase, a NADP-dependent malic enzyme, a dicarboxylic acid transporter and a ferrochelatase.

3. The method of claim 1, wherein the bovine myoglobin consists of a globin having an amino acid sequence as set forth in SEQ ID NO: 1 and a heme having formula 1:

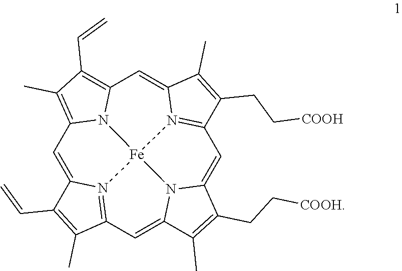

4. The method of claim 1, wherein the first plasmid has a nucleotide sequence set forth in SEQ ID NO: 6.

5. The method of claim 1, wherein the second plasmid has a nucleotide sequence set forth in SEQ ID NO: 8.

6. The method of claim 2, wherein the ALA synthase is a Rhodobacter sphaeroides ALA synthase having a nucleotide sequence set forth in SEQ ID NO: 2, the NADP-dependent malic enzyme is an Escherichia coli NADP-dependent malic enzyme having a nucleotide sequence set forth in SEQ ID NO: 3, the dicarboxylic acid transporter is an Escherichia coli dicarboxylic acid transporter having a nucleotide sequence set forth in SEQ ID NO: 4, and the ferrochelatase is an Escherichia coli ferrochelatase having a nucleotide sequence set forth in SEQ ID NO: 5.

7. The method of claim 1, further comprising:
adjusting pH to 7 to 9 using succinic acid for the culturing the first Escherichia coli production host.

8. A method for preparing a bovine myoglobin comprising:
constructing a third plasmid containing genes for heme biosynthesis pathway enzymes;
constructing a second Escherichia coli production host containing the third plasmid; and
producing the bovine myoglobin by culturing the second Escherichia coli production host.

9. The method of claim 8, wherein the heme biosynthesis pathway enzymes are an ALA synthase, a NADP-dependent malic enzyme, a dicarboxylic acid transporter and a ferrochelatase.

10. The method of claim 8, wherein the bovine myoglobin consists of a globin having an amino acid sequence as set forth in SEQ ID NO: 1 and a heme having formula 1:

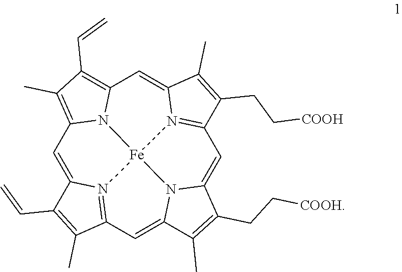

11. The method of claim 8, wherein the third plasmid has a nucleotide sequence set forth in SEQ ID NO: 9.

12. The method of claim 8, wherein the ALA synthase is a Rhodobacter sphaeroides ALA synthase having a nucleotide sequence set forth in SEQ ID NO: 2, the NADP-dependent malic enzyme is an Escherichia coli NADP-dependent malic enzyme having a nucleotide sequence set forth in SEQ ID NO: 3, the dicarboxylic acid transporter is an Escherichia coli dicarboxylic acid transporter having a nucleotide sequence set forth in SEQ ID NO: 4, and the ferrochelatase is an Escherichia coli ferrochelatase having a nucleotide sequence set forth in SEQ ID NO: 5.

13. The method of claim 8, further comprising:
adjusting pH to 7 to 9 using succinic acid for the culturing the second Escherichia coli production host.

14. A method for preparing a bovine myoglobin comprising:
constructing a second plasmid containing a gene for Bos taurus myoglobin MYG;
constructing a third Escherichia coli production host containing the second plasmid;
producing a globin by culturing the third Escherichia coli production host;
producing a heme by microbial fermentation or chemical synthesis; and
coupling of the globin and the heme to obtain the bovine myoglobin.

15. The method of claim 14, wherein the second plasmid has a nucleotide sequence set forth in SEQ ID NO: 8.

16. The method of claim 14, wherein the producing the heme comprising:
constructing a first plasmid containing genes for heme biosynthesis pathway enzymes;
constructing a fourth Escherichia coli production host containing the first plasmid; and
producing the heme by culturing the fourth Escherichia coli production host.

17. The method of claim 16, wherein the first plasmid has a nucleotide sequence set forth in SEQ ID NO: 6.

18. A composition useful as a meat flavor and/or an iron supplement, comprising the bovine myoglobin prepared in accordance with claim 1.


SEQUENCE LISTING (9 sequences)

SEQ ID NO: 1 - protein, 154 aa, Artificial Sequence
  Bovine myoglobin
MGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQAAMSKALELFRNDMAAQYKVLGFHG

SEQ ID NO: 2 - DNA, 1224 bp, Artificial Sequence
  Rhodobacter sphaeroides ALA synthase
atggactacaatctggcactcgataccgctctgaaccggctccataccgagggccggtaccggaccttcatcgacatcgagcggcgcaagggtgccttcccgaaagccatgtggcgcaagcccgacgggagcgagaaggaaatcaccgtctggtgcggcaacgactatctcggcatgggccagcatccggtggtgctgggggccatgcacgaggcgctggattcgaccggcgccgggtcgggcggcacgcgcaacatctcgggcaccacgctctatcacaagcgcctcgaggccgagctcgccgacctgcacggcaaggaagcggcgctggtcttctcgtcggcctatatcgccaacgacgcgaccctctcgacgctgccgcagctgatcccgggcctcgtcatcgtctcggacaagttgaaccacgcttcgatgatcgagggcatccgccgctcgggcaccgagaagcacatcttcaagcacaatgacctcgacgacctgcgccggatcctgacctcgatcggcaaggaccgtccgatcctcgtggccttcgaatccgtctattcgatggatggcgacttcggccgcatcgaggagatctgcgacatcgccgacgagttcggcgcgctgaaatacatcgacgaggtccatgccgtcggcatgtacggcccccgcggcggcggcgtggccgagcgggacgggctgatggaccggatcgacatcatcaacgggacgctgggcaaggcctatggcgtgttcggcggctatatcgcggcctcgtcaaagatgtgcgacgcggtgcgctcctacgcgccgggcttcatcttctcgacctcgctgccgcccgtcgtggcggccggtgcggcggcctcggtgcgccacctcaagggcgatgtggagctgcgcgagaagcaccagacccaggcccgcatcctgaagatgcgcctcaaggggctcggcctgccgatcatcgaccacggctcgcacatcgtgccggtccatgtgggcgaccccgtgcactgcaagatgatctcggacatgctgctcgagcatttcggcatctatgtccagccgatcaacttcccgaccgtgccgcgcgggaccgagcggctgcgcttcaccccgtcgcccgtgcatgattccggcatgatcgatcacctcgtgaaggccatggacgtgctctggcagcactgtgcgctgaatcgcgccgaggtcgttgcctga

SEQ ID NO: 3 - DNA, 2280 bp, Artificial Sequence
  Escherichia coli NADP-dependent malic enzyme
atggatgaccagttaaaacaaagtgcacttgatttccatgaatttccagttccagggaaaatccaggtttctccaaccaagcctctggcaacacagcgcgatctggcgctggcctactcaccaggcgttgccgcaccttgtcttgaaatcgaaaaagacccgttaaaagcctacaaatataccgcccgaggtaacctggtggcggtgatctctaacggtacggcggtgctggggttaggcaacattggcgcgctggcaggcaaaccggtgatggaaggcaagggcgttctgtttaagaaattcgccgggattgatgtatttgacattgaagttgacgaactcgacccggacaaatttattgaagttgtcgccgcgctcgaaccaaccttcggcggcatcaacctcgaagacattaaagcgccagaatgtttctatattgaacagaaactgcgcgagcggatgaatattccggtattccacgacgatcagcacggcacggcaattatcagcactgccgccatcctcaacggcttgcgcgtggtggagaaaaacatctccgacgtgcggatggtggtttccggcgcgggtgccgcagcaatcgcctgtatgaacctgctggtagcgctgggtctgcaaaaacataacatcgtggtttgcgattcaaaaggcgttatctatcagggccgtgagccaaacatggcggaaaccaaagccgcatatgcggtggtggatgacggcaaacgtaccctcgatgatgtgattgaaggcgcggatattttcctgggctgttccggcccgaaagtgctgacccaggaaatggtgaagaaaatggctcgtgcgccaatgatcctggcgctggcgaacccggaaccggaaattctgccgccgctggcgaaagaagtgcgtccggatgccatcatttgcaccggtcgttctgactatccgaaccaggtgaacaacgtcctgtgcttcccgttcatcttccgtggcgcgctggacgttggcgcaaccgccatcaacgaagagatgaaactggcggcggtacgtgcgattgcagaactcgcccatgcggaacagagcgaagtggtggcttcagcgtatggcgatcaggatctgagctttggtccggaatacatcattccaaaaccgtttgatccgcgcttgatcgttaagatcgctcctgcggtcgctaaagccgcgatggagtcgggcgtggcgactcgtccgattgctgatttcgacgtctacatcgacaagctgactgagttcgtttacaaaaccaacctgtttatgaagccgattttctcccaggctcgcaaagcgccgaagcgcgttgttctgccggaaggggaagaggcgcgcgttctgcatgccactcaggaactggtaacgctgggactggcgaaaccgatccttatcggtcgtccgaacgtgatcgaaatgcgcattcagaaactgggcttgcagatcaaagcgggcgttgattttgagatcgtcaataacgaatccgatccgcgctttaaagagtactggaccgaatacttccagatcatgaagcgtcgcggcgtcactcaggaacaggcgcagcgggcgctgatcagtaacccgacagtgatcggcgcgatcatggttcagcgtggggaagccgatgcaatgatttgcggtacggtgggtgattatcatgaacattttagcgtggtgaaaaatgtctttggttatcgcgatggcgttcacaccgcaggtgccatgaacgcgctgctgctgccgagtggtaacacctttattgccgatacatatgttaatgatgaaccggatgcagaagagctggcggagatcaccttgatggcggcagaaactgtccgtcgttttggtattgagccgcgcgttgctttgttgtcgcactccaactttggttcttctgactgcccgtcgtcgagcaaaatgcgtcaggcgctggaactggtcagggaacgtgcaccagaactgatgattgatggtgaaatgcacggcgatgcagcgctggtggaagcgattcgcaacgaccgtatgccggacagctctttgaaaggttccgccaatattctggtgatgccgaacatggaagctgcccgcattagttacaacttactgcgtgtttccagctcggaaggtgtgactgtcggcccggtgctgatgggtgtggcgaaaccggttcacgtgttaacgccgatcgcatcggtgcgtcgtatcgtcaacatggtggcgctggccgtggtagaagcgcaaacccaaccgctgtaa

SEQ ID NO: 4 - DNA, 1287 bp, Artificial Sequence
  Escherichia coli dicarboxylic acid transporter
atgaaaacctctctgtttaaaagcctttactttcaggtcctgacagcgatagccattggtattctccttggccatttctatcctgaaataggcgagcaaatgaaaccgcttggcgacggcttcgttaagctcattaagatgatcatcgctcctgtcatcttttgtaccgtcgtaacgggcattgcgggcatggaaagcatgaaggcggtcggtcgtaccggcgcagtcgcactgctttactttgaaattgtcagtaccatcgcgctgattattggtcttatcatcgttaacgtcgtgcagcctggtgccggaatgaacgtcgatccggcaacgcttgatgcgaaagcggtagcggtttacgccgatcaggcgaaagaccagggcattgtcgccttcattatggatgtcatcccggcgagcgtcattggcgcatttgccagcggtaacattctgcaggtgctgctgtttgccgtactgtttggttttgcgctccaccgtctgggcagcaaaggccaactgatttttaacgtcatcgaaagtttctcgcaggtcatcttcggcatcatcaatatgatcatgcgtctggcacctattggtgcgttcggggcaatggcgtttaccatcggtaaatacggcgtcggcacactggtgcaactggggcagctgattatctgtttctacattacctgtatcctgtttgtggtgctggtattgggttcaatcgctaaagcgactggtttcagtatcttcaaatttatccgctacatccgtgaagaactgctgattgtactggggacttcatcttccgagtcggcgctgccgcgtatgctcgacaagatggagaaactcggctgccgtaaatcggtggtggggctggtcatcccgacaggctactcgtttaaccttgatggcacatcgatatacctgacaatggcggcggtgtttatcgcccaggccactaacagtcagatggatatcgtccaccaaatcacgctgttaatcgtgttgctgctttcttctaaaggggcggcaggggtaacgggtagtggctttatcgtgctggcggcgacgctctctgcggtgggccatttgccggtagcgggtctggcgctgatcctcggtatcgaccgctttatgtcagaagctcgtgcgctgactaacctggtcggtaacggcgtagcgaccattgtcgttgctaagtgggtgaaagaactggaccacaaaaaactggacgatgtgctgaataatcgtgcgccggatggcaaaacgcacgaattatcctcttaa

SEQ ID NO: 5 - DNA, 963 bp, Artificial Sequence
  Escherichia coli ferrochelatase
atgcgtcagactaaaaccggtatcctgctggcaaacctgggtacgcccgatgcccccacacctgaagcggtaaaacgctatctgaaacaatttttaagcgacagacgcgtggttgatacctcacggttgttatggtggccattgctgcgcggcgtgattttgccgctgcgctcgccgcgtgtggcgaagctgtatgcctctgtctggatggaaggtggctcgccgctgatggtttacagccgccagcaacagcaggcgctggcacaacgtttaccggagatgcccgtagcgctgggaatgagctacggctcgccatcactggaaagcgccgtagatgaactcctggcagagcatgtagatcatattgtggtgctgccgctttatccgcaattctcctgttctacggtcggtgcggtatgggatgaactggcacgcattctggcgcgcaaacgtagcattccggggatatcgtttatacgtgattacgccgataaccacgattacattaatgcactggcgaacagcgtacgcgcttcttttgccaaacatggcgaaccggatctgctactgctctcttatcatggcattccccagcgttatgcagatgaaggcgatgattacccgcaacgttgccgcacaacgactcgtgaactggcttccgcattggggatggcaccggaaaaagtgatgatgacctttcagtcgcgctttggtcgggaaccctggctgatgccttataccgacgaaacgctgaaaatgctcggagaaaaaggcgtaggtcatattcaggtgatgtgcccgggctttgctgcggattgtctggagacgctggaagagattgccgagcaaaaccgtgaggtcttcctcggtgccggcgggaaaaaatatgaatatattccggcgcttaatgccacgccggaacatatcgaaatgatggctaatcttgttgccgcgtatcgctaa

SEQ ID NO: 6 - DNA, 9912 bp, Artificial Sequence
  Plasmid pLEX_HMDH
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtcgctgccgcgccggtagtacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggatccccctcaagtcaaaagcctccggtcggaggcttttgactttctgctatggaggtcaggtatgatttttatgacaacttgacggctacatcattcactttttcttcacaaccggcacggaactcgctcgggctggccccgctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagaattgatctctcacctaccaaacaatgcccccctgcaaaaaataaattcatataaaaaacatacagataaccatctgcggtgataaattatctctggcggtgttgacataaataccactggcggtgatactgagcacatcagcaggacgcactgaccaccatgaaggtgacgctcttaaaaattaagccctgaagaagggctttatttgcatacattcaatcaattgttatctaaggaaatacttacatatggactacaatctggcactcgataccgctctgaaccggctccataccgagggccggtaccggaccttcatcgacatcgagcggcgcaagggtgccttcccgaaagccatgtggcgcaagcccgacgggagcgagaaggaaatcaccgtctggtgcggcaacgactatctcggcatgggccagcatccggtggtgctgggggccatgcacgaggcgctggattcgaccggcgccgggtcgggcggcacgcgcaacatctcgggcaccacgctctatcacaagcgcctcgaggccgagctcgccgacctgcacggcaaggaagcggcgctggtcttctcgtcggcctatatcgccaacgacgcgaccctctcgacgctgccgcagctgatcccgggcctcgtcatcgtctcggacaagttgaaccacgcttcgatgatcgagggcatccgccgctcgggcaccgagaagcacatcttcaagcacaatgacctcgacgacctgcgccggatcctgacctcgatcggcaaggaccgtccgatcctcgtggccttcgaatccgtctattcgatggatggcgacttcggccgcatcgaggagatctgcgacatcgccgacgagttcggcgcgctgaaatacatcgacgaggtccatgccgtcggcatgtacggcccccgcggcggcggcgtggccgagcgggacgggctgatggaccggatcgacatcatcaacgggacgctgggcaaggcctatggcgtgttcggcggctatatcgcggcctcgtcaaagatgtgcgacgcggtgcgctcctacgcgccgggcttcatcttctcgacctcgctgccgcccgtcgtggcggccggtgcggcggcctcggtgcgccacctcaagggcgatgtggagctgcgcgagaagcaccagacccaggcccgcatcctgaagatgcgcctcaaggggctcggcctgccgatcatcgaccacggctcgcacatcgtgccggtccatgtgggcgaccccgtgcactgcaagatgatctcggacatgctgctcgagcatttcggcatctatgtccagccgatcaacttcccgaccgtgccgcgcgggaccgagcggctgcgcttcaccccgtcgcccgtgcatgattccggcatgatcgatcacctcgtgaaggccatggacgtgctctggcagcactgtgcgctgaatcgcgccgaggtcgttgcctgacagcttctgcggatgcaaaggcccctgccctgtgctacttctttcgggacagggcacccctgagtcggaagcaaccggccggggtaaatcggggcaggacgggcacacgcatgatctggcggaggacacaaccttcgacggccgaagtcgataaacccaaagggttcgacgatttcgagttgcggttgggcgacctgatgcgcggtgagcgggcgacgctcggcaagtcgctgctcgatgtccagcgcgagctgaagatcaaggccacctatatcgccgccatcgagaatgccgacgtgtcggccttcgagacgcagggcttcgtggcgggatatgtgcgctcctatgcgcgctatctcggcatggacccggacgaggccttcgcgcgcttctgccacgaggcgaacttcaccacgatgcacggcatggccgtttcggtgaccggcgcgcgccgcgataccggtccgcggtcccgaccgcagggcgaggggcgcgatccgctggcggatccgtcgacctgcagtaatcgtacagggtagtacaaataaaaaaggcacgtcagatgacgtgccttttttcttgtgagcagtaagcttactagtcggtgataaattatctctggcggtgttgacataaataccactggcggtgatactgagcacatcagcaggacgcactgaccaccatgaaggtgacgctcttaaaaattaagccctgaagaagggctttatttgcatacattcaatcaattgttatctaaggaaatacttacatatggatgaccagttaaaacaaagtgcacttgatttccatgaatttccagttccagggaaaatccaggtttctccaaccaagcctctggcaacacagcgcgatctggcgctggcctactcaccaggcgttgccgcaccttgtcttgaaatcgaaaaagacccgttaaaagcctacaaatataccgcccgaggtaacctggtggcggtgatctctaacggtacggcggtgctggggttaggcaacattggcgcgctggcaggcaaaccggtgatggaaggcaagggcgttctgtttaagaaattcgccgggattgatgtatttgacattgaagttgacgaactcgacccggacaaatttattgaagttgtcgccgcgctcgaaccaaccttcggcggcatcaacctcgaagacattaaagcgccagaatgtttctatattgaacagaaactgcgcgagcggatgaatattccggtattccacgacgatcagcacggcacggcaattatcagcactgccgccatcctcaacggcttgcgcgtggtggagaaaaacatctccgacgtgcggatggtggtttccggcgcgggtgccgcagcaatcgcctgtatgaacctgctggtagcgctgggtctgcaaaaacataacatcgtggtttgcgattcaaaaggcgttatctatcagggccgtgagccaaacatggcggaaaccaaagccgcatatgcggtggtggatgacggcaaacgtaccctcgatgatgtgattgaaggcgcggatattttcctgggctgttccggcccgaaagtgctgacccaggaaatggtgaagaaaatggctcgtgcgccaatgatcctggcgctggcgaacccggaaccggaaattctgccgccgctggcgaaagaagtgcgtccggatgccatcatttgcaccggtcgttctgactatccgaaccaggtgaacaacgtcctgtgcttcccgttcatcttccgtggcgcgctggacgttggcgcaaccgccatcaacgaagagatgaaactggcggcggtacgtgcgattgcagaactcgcccatgcggaacagagcgaagtggtggcttcagcgtatggcgatcaggatctgagctttggtccggaatacatcattccaaaaccgtttgatccgcgcttgatcgttaagatcgctcctgcggtcgctaaagccgcgatggagtcgggcgtggcgactcgtccgattgctgatttcgacgtctacatcgacaagctgactgagttcgtttacaaaaccaacctgtttatgaagccgattttctcccaggctcgcaaagcgccgaagcgcgttgttctgccggaaggggaagaggcgcgcgttctgcatgccactcaggaactggtaacgctgggactggcgaaaccgatccttatcggtcgtccgaacgtgatcgaaatgcgcattcagaaactgggcttgcagatcaaagcgggcgttgattttgagatcgtcaataacgaatccgatccgcgctttaaagagtactggaccgaatacttccagatcatgaagcgtcgcggcgtcactcaggaacaggcgcagcgggcgctgatcagtaacccgacagtgatcggcgcgatcatggttcagcgtggggaagccgatgcaatgatttgcggtacggtgggtgattatcatgaacattttagcgtggtgaaaaatgtctttggttatcgcgatggcgttcacaccgcaggtgccatgaacgcgctgctgctgccgagtggtaacacctttattgccgatacatatgttaatgatgaaccggatgcagaagagctggcggagatcaccttgatggcggcagaaactgtccgtcgttttggtattgagccgcgcgttgctttgttgtcgcactccaactttggttcttctgactgcccgtcgtcgagcaaaatgcgtcaggcgctggaactggtcagggaacgtgcaccagaactgatgattgatggtgaaatgcacggcgatgcagcgctggtggaagcgattcgcaacgaccgtatgccggacagctctttgaaaggttccgccaatattctggtgatgccgaacatggaagctgcccgcattagttacaacttactgcgtgtttccagctcggaaggtgtgactgtcggcccggtgctgatgggtgtggcgaaaccggttcacgtgttaacgccgatcgcatcggtgcgtcgtatcgtcaacatggtggcgctggccgtggtagaagcgcaaacccaaccgctgtaagtcgacctgcagtaatcgtacagggtagtacaaataaaaaaggcacgtcagatgacgtgccttttttcttgtgagcagtaagcttgaattccggtgataaattatctctggcggtgttgacataaataccactggcggtgatactgagcacatcagcaggacgcactgaccaccatgaaggtgacgctcttaaaaattaagccctgaagaagggctttatttgcatacattcaatcaattgttatctaaggaaatacttacatatgaaaacctctctgtttaaaagcctttactttcaggtcctgacagcgatagccattggtattctccttggccatttctatcctgaaataggcgagcaaatgaaaccgcttggcgacggcttcgttaagctcattaagatgatcatcgctcctgtcatcttttgtaccgtcgtaacgggcattgcgggcatggaaagcatgaaggcggtcggtcgtaccggcgcagtcgcactgctttactttgaaattgtcagtaccatcgcgctgattattggtcttatcatcgttaacgtcgtgcagcctggtgccggaatgaacgtcgatccggcaacgcttgatgcgaaagcggtagcggtttacgccgatcaggcgaaagaccagggcattgtcgccttcattatggatgtcatcccggcgagcgtcattggcgcatttgccagcggtaacattctgcaggtgctgctgtttgccgtactgtttggttttgcgctccaccgtctgggcagcaaaggccaactgatttttaacgtcatcgaaagtttctcgcaggtcatcttcggcatcatcaatatgatcatgcgtctggcacctattggtgcgttcggggcaatggcgtttaccatcggtaaatacggcgtcggcacactggtgcaactggggcagctgattatctgtttctacattacctgtatcctgtttgtggtgctggtattgggttcaatcgctaaagcgactggtttcagtatcttcaaatttatccgctacatccgtgaagaactgctgattgtactggggacttcatcttccgagtcggcgctgccgcgtatgctcgacaagatggagaaactcggctgccgtaaatcggtggtggggctggtcatcccgacaggctactcgtttaaccttgatggcacatcgatatacctgacaatggcggcggtgtttatcgcccaggccactaacagtcagatggatatcgtccaccaaatcacgctgttaatcgtgttgctgctttcttctaaaggggcggcaggggtaacgggtagtggctttatcgtgctggcggcgacgctctctgcggtgggccatttgccggtagcgggtctggcgctgatcctcggtatcgaccgctttatgtcagaagctcgtgcgctgactaacctggtcggtaacggcgtagcgaccattgtcgttgctaagtgggtgaaagaactggaccacaaaaaactggacgatgtgctgaataatcgtgcgccggatggcaaaacgcacgaattatcctcttaagtcgacctgcagtaatcgtacagggtagtacaaataaaaaaggcacgtcagatgacgtgccttttttcttgtgagcagtaagcttgcggccgccggtgataaattatctctggcggtgttgacataaataccactggcggtgatactgagcacatcagcaggacgcactgaccaccatgaaggtgacgctcttaaaaattaagccctgaagaagggctttatttgcatacattcaatcaattgttatctaaggaaatacttacatatgcgtcagactaaaaccggtatcctgctggcaaacctgggtacgcccgatgcccccacacctgaagcggtaaaacgctatctgaaacaatttttaagcgacagacgcgtggttgatacctcacggttgttatggtggccattgctgcgcggcgtgattttgccgctgcgctcgccgcgtgtggcgaagctgtatgcctctgtctggatggaaggtggctcgccgctgatggtttacagccgccagcaacagcaggcgctggcacaacgtttaccggagatgcccgtagcgctgggaatgagctacggctcgccatcactggaaagcgccgtagatgaactcctggcagagcatgtagatcatattgtggtgctgccgctttatccgcaattctcctgttctacggtcggtgcggtatgggatgaactggcacgcattctggcgcgcaaacgtagcattccggggatatcgtttatacgtgattacgccgataaccacgattacattaatgcactggcgaacagcgtacgcgcttcttttgccaaacatggcgaaccggatctgctactgctctcttatcatggcattccccagcgttatgcagatgaaggcgatgattacccgcaacgttgccgcacaacgactcgtgaactggcttccgcattggggatggcaccggaaaaagtgatgatgacctttcagtcgcgctttggtcgggaaccctggctgatgccttataccgacgaaacgctgaaaatgctcggagaaaaaggcgtaggtcatattcaggtgatgtgcccgggctttgctgcggattgtctggagacgctggaagagattgccgagcaaaaccgtgaggtcttcctcggtgccggcgggaaaaaatatgaatatattccggcgcttaatgccacgccggaacatatcgaaatgatggctaatcttgttgccgcgtatcgctaaactagtgtcgacctgcagtaatcgtacagggtagtacaaataaaaaaggcacgtcagatgacgtgccttttttcttgtgagcagtaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgc

SEQ ID NO: 7 - DNA, 465 bp, Artificial Sequence
  Bos taurus myoglobin MYG
atgggcttgtcggatggtgaatggcaactggtcctgaacgcctggggtaaagttgaagcggacgttgctgggcacggacaggaagtactgatccgtttgttcactgggcatcctgaaacactggaaaagtttgacaaattcaaacacttgaagaccgaagcggaaatgaaggcttcggaagacctgaagaagcacggaaatacagtcttgacggctttggggggaatcctgaagaaaaagggtcaccatgaggcagaggtaaaacatttagccgagagtcacgcgaataaacataagattcccgtgaagtatctggaatttattagcgacgccattatccatgtgttgcacgcaaaacatccaagcgactttggagccgatgcacaggcggcaatgtcgaaagctcttgaattattccgtaatgacatggcggcacagtacaaagtgcttggctttcacggataa

SEQ ID NO: 8 - DNA, 4445 bp, Artificial Sequence
  Plasmid pBAD_BMYG
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagaaataattttgtttaactttaagaaggagatatacatccatgggcttgtcggatggtgaatggcaactggtcctgaacgcctggggtaaagttgaagcggacgttgctgggcacggacaggaagtactgatccgtttgttcactgggcatcctgaaacactggaaaagtttgacaaattcaaacacttgaagaccgaagcggaaatgaaggcttcggaagacctgaagaagcacggaaatacagtcttgacggctttggggggaatcctgaagaaaaagggtcaccatgaggcagaggtaaaacatttagccgagagtcacgcgaataaacataagattcccgtgaagtatctggaatttattagcgacgccattatccatgtgttgcacgcaaaacatccaagcgactttggagccgatgcacaggcggcaatgtcgaaagctcttgaattattccgtaatgacatggcggcacagtacaaagtgcttggctttcacggataagcggccgcgtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagattatcaaaaaggatcttcacctagatccttttaaattaaaaatgaagttttaaatcaatctaaagtatatatgagtaaacttggtctgacagttaggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtcagttgtgcccagtcatagccgaatagcctctccacccaagcggccggagaacctgcgtgcaatccatcttgttcaatcatactcttcctttttcaatattattgaagcatttatcagggttattgtctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagcagatcaattcgcgcgcgaaggcgaagcggcatgcataatgtgcctgtcaaatggacgaagcagggattctgcaaaccctatgctactccgtcaagccgtcaattgtctgattcgttaccaattatgacaacttgacggctacatcattcactttttcttcacaaccggcacggaactcgctcgggctggccccggtgcattttttaaatacccgcgagaaatagagttgatcgtcaaaaccaacattgcgaccgacggtggcgataggcatccgggtggtgctcaaaagcagcttcgcctggctgatacgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaccccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcaaacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcattaaacgagtatcccggcagcaggggatcattttgcgcttcagccatacttttcatactcccgccattcagag

SEQ ID NO: 9 - DNA, 10785 bp, Artificial Sequence
  Plasmid pLEX_BHMDH
gacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtcgctgccgcgccggtagtacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaacgcgtggatccccctcaagtcaaaagcctccggtcggaggcttttgactttctgctatggaggtcaggtatgatttttatgacaacttgacggctacatcattcactttttcttcacaaccggcacggaactcgctcgggctggccccgctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttttatccataagattagcggatcctacctgacgctttttatcgcaactctctactgtttctccatacccgtttttttgggctagaaataattttgtttaactttaagaaggagatatacatccatgggcttgtcggatggtgaatggcaactggtcctgaacgcctggggtaaagttgaagcggacgttgctgggcacggacaggaagtactgatccgtttgttcactgggcatcctgaaacactggaaaagtttgacaaattcaaacacttgaagaccgaagcggaaatgaaggcttcggaagacctgaagaagcacggaaatacagtcttgacggctttggggggaatcctgaagaaaaagggtcaccatgaggcagaggtaaaacatttagccgagagtcacgcgaataaacataagattcccgtgaagtatctggaatttattagcgacgccattatccatgtgttgcacgcaaaacatccaagcgactttggagccgatgcacaggcggcaatgtcgaaagctcttgaattattccgtaatgacatggcggcacagtacaaagtgcttggctttcacggataagcggccgcgtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagaattgatctctcacctaccaaacaatgcccccctgcaaaaaataaattcatataaaaaacatacagataaccatctgcggtgataaattatctctggcggtgttgacataaataccactggcggtgatactgagcacatcagcaggacgcactgaccaccatgaaggtgacgctcttaaaaattaagccctgaagaagggctttatttgcatacattcaatcaattgttatctaaggaaatacttacatatggactacaatctggcactcgataccgctctgaaccggctccataccgagggccggtaccggaccttcatcgacatcgagcggcgcaagggtgccttcccgaaagccatgtggcgcaagcccgacgggagcgagaaggaaatcaccgtctggtgcggcaacgactatctcggcatgggccagcatccggtggtgctgggggccatgcacgaggcgctggattcgaccggcgccgggtcgggcggcacgcgcaacatctcgggcaccacgctctatcacaagcgcctcgaggccgagctcgccgacctgcacggcaaggaagcggcgctggtcttctcgtcggcctatatcgccaacgacgcgaccctctcgacgctgccgcagctgatcccgggcctcgtcatcgtctcggacaagttgaaccacgcttcgatgatcgagggcatccgccgctcgggcaccgagaagcacatcttcaagcacaatgacctcgacgacctgcgccggatcctgacctcgatcggcaaggaccgtccgatcctcgtggccttcgaatccgtctattcgatggatggcgacttcggccgcatcgaggagatctgcgacatcgccgacgagttcggcgcgctgaaatacatcgacgaggtccatgccgtcggcatgtacggcccccgcggcggcggcgtggccgagcgggacgggctgatggaccggatcgacatcatcaacgggacgctgggcaaggcctatggcgtgttcggcggctatatcgcggcctcgtcaaagatgtgcgacgcggtgcgctcctacgcgccgggcttcatcttctcgacctcgctgccgcccgtcgtggcggccggtgcggcggcctcggtgcgccacctcaagggcgatgtggagctgcgcgagaagcaccagacccaggcccgcatcctgaagatgcgcctcaaggggctcggcctgccgatcatcgaccacggctcgcacatcgtgccggtccatgtgggcgaccccgtgcactgcaagatgatctcggacatgctgctcgagcatttcggcatctatgtccagccgatcaacttcccgaccgtgccgcgcgggaccgagcggctgcgcttcaccccgtcgcccgtgcatgattccggcatgatcgatcacctcgtgaaggccatggacgtgctctggcagcactgtgcgctgaatcgcgccgaggtcgttgcctgacagcttctgcggatgcaaaggcccctgccctgtgctacttctttcgggacagggcacccctgagtcggaagcaaccggccggggtaaatcggggcaggacgggcacacgcatgatctggcggaggacacaaccttcgacggccgaagtcgataaacccaaagggttcgacgatttcgagttgcggttgggcgacctgatgcgcggtgagcgggcgacgctcggcaagtcgctgctcgatgtccagcgcgagctgaagatcaaggccacctatatcgccgccatcgagaatgccgacgtgtcggccttcgagacgcagggcttcgtggcgggatatgtgcgctcctatgcgcgctatctcggcatggacccggacgaggccttcgcgcgcttctgccacgaggcgaacttcaccacgatgcacggcatggccgtttcggtgaccggcgcgcgccgcgataccggtccgcggtcccgaccgcagggcgaggggcgcgatccgctggcggatccgtcgacctgcagtaatcgtacagggtagtacaaataaaaaaggcacgtcagatgacgtgccttttttcttgtgagcagtaagcttactagtcggtgataaattatctctggcggtgttgacataaataccactggcggtgatactgagcacatcagcaggacgcactgaccaccatgaaggtgacgctcttaaaaattaagccctgaagaagggctttatttgcatacattcaatcaattgttatctaaggaaatacttacatatggatgaccagttaaaacaaagtgcacttgatttccatgaatttccagttccagggaaaatccaggtttctccaaccaagcctctggcaacacagcgcgatctggcgctggcctactcaccaggcgttgccgcaccttgtcttgaaatcgaaaaagacccgttaaaagcctacaaatataccgcccgaggtaacctggtggcggtgatctctaacggtacggcggtgctggggttaggcaacattggcgcgctggcaggcaaaccggtgatggaaggcaagggcgttctgtttaagaaattcgccgggattgatgtatttgacattgaagttgacgaactcgacccggacaaatttattgaagttgtcgccgcgctcgaaccaaccttcggcggcatcaacctcgaagacattaaagcgccagaatgtttctatattgaacagaaactgcgcgagcggatgaatattccggtattccacgacgatcagcacggcacggcaattatcagcactgccgccatcctcaacggcttgcgcgtggtggagaaaaacatctccgacgtgcggatggtggtttccggcgcgggtgccgcagcaatcgcctgtatgaacctgctggtagcgctgggtctgcaaaaacataacatcgtggtttgcgattcaaaaggcgttatctatcagggccgtgagccaaacatggcggaaaccaaagccgcatatgcggtggtggatgacggcaaacgtaccctcgatgatgtgattgaaggcgcggatattttcctgggctgttccggcccgaaagtgctgacccaggaaatggtgaagaaaatggctcgtgcgccaatgatcctggcgctggcgaacccggaaccggaaattctgccgccgctggcgaaagaagtgcgtccggatgccatcatttgcaccggtcgttctgactatccgaaccaggtgaacaacgtcctgtgcttcccgttcatcttccgtggcgcgctggacgttggcgcaaccgccatcaacgaagagatgaaactggcggcggtacgtgcgattgcagaactcgcccatgcggaacagagcgaagtggtggcttcagcgtatggcgatcaggatctgagctttggtccggaatacatcattccaaaaccgtttgatccgcgcttgatcgttaagatcgctcctgcggtcgctaaagccgcgatggagtcgggcgtggcgactcgtccgattgctgatttcgacgtctacatcgacaagctgactgagttcgtttacaaaaccaacctgtttatgaagccgattttctcccaggctcgcaaagcgccgaagcgcgttgttctgccggaaggggaagaggcgcgcgttctgcatgccactcaggaactggtaacgctgggactggcgaaaccgatccttatcggtcgtccgaacgtgatcgaaatgcgcattcagaaactgggcttgcagatcaaagcgggcgttgattttgagatcgtcaataacgaatccgatccgcgctttaaagagtactggaccgaatacttccagatcatgaagcgtcgcggcgtcactcaggaacaggcgcagcgggcgctgatcagtaacccgacagtgatcggcgcgatcatggttcagcgtggggaagccgatgcaatgatttgcggtacggtgggtgattatcatgaacattttagcgtggtgaaaaatgtctttggttatcgcgatggcgttcacaccgcaggtgccatgaacgcgctgctgctgccgagtggtaacacctttattgccgatacatatgttaatgatgaaccggatgcagaagagctggcggagatcaccttgatggcggcagaaactgtccgtcgttttggtattgagccgcgcgttgctttgttgtcgcactccaactttggttcttctgactgcccgtcgtcgagcaaaatgcgtcaggcgctggaactggtcagggaacgtgcaccagaactgatgattgatggtgaaatgcacggcgatgcagcgctggtggaagcgattcgcaacgaccgtatgccggacagctctttgaaaggttccgccaatattctggtgatgccgaacatggaagctgcccgcattagttacaacttactgcgtgtttccagctcggaaggtgtgactgtcggcccggtgctgatgggtgtggcgaaaccggttcacgtgttaacgccgatcgcatcggtgcgtcgtatcgtcaacatggtggcgctggccgtggtagaagcgcaaacccaaccgctgtaagtcgacctgcagtaatcgtacagggtagtacaaataaaaaaggcacgtcagatgacgtgccttttttcttgtgagcagtaagcttgaattccggtgataaattatctctggcggtgttgacataaataccactggcggtgatactgagcacatcagcaggacgcactgaccaccatgaaggtgacgctcttaaaaattaagccctgaagaagggctttatttgcatacattcaatcaattgttatctaaggaaatacttacatatgaaaacctctctgtttaaaagcctttactttcaggtcctgacagcgatagccattggtattctccttggccatttctatcctgaaataggcgagcaaatgaaaccgcttggcgacggcttcgttaagctcattaagatgatcatcgctcctgtcatcttttgtaccgtcgtaacgggcattgcgggcatggaaagcatgaaggcggtcggtcgtaccggcgcagtcgcactgctttactttgaaattgtcagtaccatcgcgctgattattggtcttatcatcgttaacgtcgtgcagcctggtgccggaatgaacgtcgatccggcaacgcttgatgcgaaagcggtagcggtttacgccgatcaggcgaaagaccagggcattgtcgccttcattatggatgtcatcccggcgagcgtcattggcgcatttgccagcggtaacattctgcaggtgctgctgtttgccgtactgtttggttttgcgctccaccgtctgggcagcaaaggccaactgatttttaacgtcatcgaaagtttctcgcaggtcatcttcggcatcatcaatatgatcatgcgtctggcacctattggtgcgttcggggcaatggcgtttaccatcggtaaatacggcgtcggcacactggtgcaactggggcagctgattatctgtttctacattacctgtatcctgtttgtggtgctggtattgggttcaatcgctaaagcgactggtttcagtatcttcaaatttatccgctacatccgtgaagaactgctgattgtactggggacttcatcttccgagtcggcgctgccgcgtatgctcgacaagatggagaaactcggctgccgtaaatcggtggtggggctggtcatcccgacaggctactcgtttaaccttgatggcacatcgatatacctgacaatggcggcggtgtttatcgcccaggccactaacagtcagatggatatcgtccaccaaatcacgctgttaatcgtgttgctgctttcttctaaaggggcggcaggggtaacgggtagtggctttatcgtgctggcggcgacgctctctgcggtgggccatttgccggtagcgggtctggcgctgatcctcggtatcgaccgctttatgtcagaagctcgtgcgctgactaacctggtcggtaacggcgtagcgaccattgtcgttgctaagtgggtgaaagaactggaccacaaaaaactggacgatgtgctgaataatcgtgcgccggatggcaaaacgcacgaattatcctcttaagtcgacctgcagtaatcgtacagggtagtacaaataaaaaaggcacgtcagatgacgtgccttttttcttgtgagcagtaagcttgcggccgccggtgataaattatctctggcggtgttgacataaataccactggcggtgatactgagcacatcagcaggacgcactgaccaccatgaaggtgacgctcttaaaaattaagccctgaagaagggctttatttgcatacattcaatcaattgttatctaaggaaatacttacatatgcgtcagactaaaaccggtatcctgctggcaaacctgggtacgcccgatgcccccacacctgaagcggtaaaacgctatctgaaacaatttttaagcgacagacgcgtggttgatacctcacggttgttatggtggccattgctgcgcggcgtgattttgccgctgcgctcgccgcgtgtggcgaagctgtatgcctctgtctggatggaaggtggctcgccgctgatggtttacagccgccagcaacagcaggcgctggcacaacgtttaccggagatgcccgtagcgctgggaatgagctacggctcgccatcactggaaagcgccgtagatgaactcctggcagagcatgtagatcatattgtggtgctgccgctttatccgcaattctcctgttctacggtcggtgcggtatgggatgaactggcacgcattctggcgcgcaaacgtagcattccggggatatcgtttatacgtgattacgccgataaccacgattacattaatgcactggcgaacagcgtacgcgcttcttttgccaaacatggcgaaccggatctgctactgctctcttatcatggcattccccagcgttatgcagatgaaggcgatgattacccgcaacgttgccgcacaacgactcgtgaactggcttccgcattggggatggcaccggaaaaagtgatgatgacctttcagtcgcgctttggtcgggaaccctggctgatgccttataccgacgaaacgctgaaaatgctcggagaaaaaggcgtaggtcatattcaggtgatgtgcccgggctttgctgcggattgtctggagacgctggaagagattgccgagcaaaaccgtgaggtcttcctcggtgccggcgggaaaaaatatgaatatattccggcgcttaatgccacgccggaacatatcgaaatgatggctaatcttgttgccgcgtatcgctaaactagtgtcgacctgcagtaatcgtacagggtagtacaaataaaaaaggcacgtcagatgacgtgccttttttcttgtgagcagtaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgc